(19) United States
(12) Patent Application Publication
(10) Pub. No.: US 2026/0357235 A1
(43) Pub. Date: Nov. 12, 2026

(54) CULTURE MEDIUM FOR CULTIVATING HATHEWAYA HISTOLYTICA (OR CLOSTRIDIUM HISTOLYTICUM) AND THE PRODUCTION OF ONE OR MORE PROTEASES

(71) Applicant: NORDMARK PHARMA GMBH, Uetersen (DE)

(72) Inventors: Jan HEYLAND, Uetersen (DE); Malgorzata STAWORZYNSKA-GÖDDE, Uetersen (DE); Thomas SCHRÄDER, Uetersen (DE)

(73) Assignee: NORDMARK PHARMA GMBH, Uetersen (DE)

(21) Appl. No.: 18/707,669

(22) PCT Filed: Jan. 2, 2023
(86) PCT No.: PCT/EP2023/050027

§ 371 Date: May 6, 2024

(30) Foreign Application Priority Data
Jan. 5, 2022 (EP) 22150333.7
Aug. 30, 2022 (DE) 10 2022 121 862.7

(51) Int. Cl. C12N 9/52; C12N 1/20

(52) U.S. Cl. CPC C12N 9/52; C12N 1/20; C12Y 304/22008; C12Y 304/24003

(57) ABSTRACT

The present invention relates to a liquid culture medium comprising yeast extract, glycine, arginine, glutamine, serine, threonine, magnesium ions, calcium ions, selenium and water. It further relates to a liquid feed composition for use as feed in a fed-batch process comprising at least yeast extract, threonine, serine and water. It relates to a kit of parts comprising the liquid culture medium and the liquid feed composition, the use of the composition for cultivating Hathewaya histolytica (Clostridium histolyticum) and obtaining one or more proteases from the culture supernatant and methods concerning the cultivation.

### INTRODUCTION

The present invention relates to a liquid culture medium comprising yeast extract, glycine, arginine, glutamine, serine, threonine, magnesium ions, calcium ions, selenium (e.g., in the form of sodium selenite) and water. It further relates to a liquid feed composition for use as feed in a fed-batch process comprising at least yeast extract, threonine, serine and water. It relates to a kit of parts comprising the liquid culture medium and the liquid feed composition, the use of the liquid culture medium and the liquid feed composition for cultivating Hathewaya histolytica (previously Clostridium histolyticum) and obtaining one or more proteases and methods concerning the cultivation.

### DISCUSSION OF PRIOR ART

Collagen is the major structural constituent of mammalian organisms and makes up a large portion of the total protein content of skin and other parts of the animal body. In humans, it is particularly important in the wound healing process and in the process of natural aging. Various skin traumas such as bumps, surgery, infection and accident are often characterized by the erratic accumulation of fibrous tissue that is rich in collagen and having increased proteoglycan content. In addition to the replacement of the normal tissue which has been damaged or destroyed, excessive and disfiguring deposits of new tissue sometimes form during the healing process.

Numerous diseases and conditions are associated with excess collagen deposition and the erratic accumulation of fibrous tissue rich in collagen. Such diseases and conditions are collectively referred to herein as “collagen-mediated diseases”. Collagenase is an enzyme that has the specific ability to digest collagen. Collagenase has been used to treat a variety of collagen-mediated diseases such as Peyronie's disease and Dupuytren's disease, and to remove necrotic tissue from wounds.

Furthermore, collagenase and other proteases (such as e.g. neutral protease and/or clostripain) are used in vitro for tissue dissociation and isolation of a variety of cells, such as e.g. pancreatic islet cells, hepatocytes and tumor cells. These cells find multiple applications in research and clinics. One common source of crude collagenase or mixtures of proteases is from a bacterial fermentation process, especially from the fermentation of Hathewaya histolytica (previously Clostridium histolyticum).

Hathewaya (Clostridium) is a genus of gram-positive bacteria. Hathewaya (Clostridium) bacteria are anaerobic and are common in the soil, water and in the intestinal tracts of humans and other animals. The species Hathewaya histolytica (Clostridium histolyticum) is capable of producing collagen degrading enzymes and other enzymes with proteolytic activity, such as e.g. collagenases (EC 3.4.24.3), neutral protease and clostripain (EC 3.4.22.8). Important products of the cultivation process are the bacterial collagenases which possess proteolytic activity against collagen. These enzymes have been classified by Bond et al (Bond, M. D., van Wart, H. E.; Biochemistry, 23, 3077-91 (1984)) as type I and II collagenases (hereafter “collagenase I” and “collagenase II”) according to their relative activity. The collagenases and the other proteases are secreted into the culture medium and can be obtained from the culture supernatant.

In the biotech industry a number of enzymes for use in pharmaceutical applications are produced in large-scale fermentation processes. For example, Hathewaya histolytica (Clostridium histolyticum) is cultivated to produce proteases, including e.g. collagenases, neutral protease and/or clostripain. Usually, in such processes the culture media (nutrient media, fermentation media) comprise animal-derived components such as meat (tissue) peptones of either bovine or porcine origin. Because adventitious agents (e.g. viruses) originating from animal-derived growth media might be present in the final product, it is desirable to develop culture media free of mammalian pathogenic agents. There is a particular safety-related concern regarding prions and BSE. In recent years, regulatory authorities request these concerns to be addressed. Therefore, there is a tendency in the industry towards animal-free culture media.

In EP 2 133 415 A1 a growth medium for Hathewaya histolytica (Clostridium histolyticum) is disclosed that comprises water, fish gelatin and a peptone from a non-mammalian source, whereby fish peptone is excluded. The only examples for a peptone from a non-mammalian source are plant peptones. This growth medium suffers from the fact that fish gelatin and a peptone from a non-mammalian source are complex ingredients that comprise a large variety of compounds, most of which are not known. Moreover, they are prepared from natural sources and are therefore subject to natural fluctuations in their contents.

Yeast extracts and plant peptones have recently been considered as substitutes for animal-derived culture media components. However, yeast extracts alone may not provide all nutrients necessary for sufficient growth of certain bacteria. EP 2 865 748 A1 discloses animal product-free culture media for bacteria of the genus Clostridium (now Hathewaya), especially Clostridium histolyticum (now Hathewaya histolytica) comprising water, non-animal origin peptone, or its derivatives, yeast extract and the amino acids cysteine and arginine. The non-animal origin peptones are derived from plants (plant peptones). Vegetable peptones are used according to WO 2007/089851 A1 for the fermentation of Clostridium histolyticum (now Hathewaya histolytica) to produce collagenase I and collagenase II. Plant peptones have the disadvantages that they are complex and undefined mixtures and that the production of the raw materials is often subject to large batch variability, especially if weather conditions or planting regions change. Moreover, the composition of plant peptones may significantly vary depending on the production process and the supplier. Thus, it is difficult or just not possible to obtain a constant product quality or to find alternative suppliers. For these reasons, plant peptones are not desired as ingredient for most industrial processes for the cultivation of

bacteria. This is especially true for the cultivation of Hathewaya histolytica (Clostridium histolyticum) and the production of proteases. Hathewaya histolytica (Clostridium histolyticum) secretes collagenase I, collagenase II, clostripain and neutral protease during cultivation. The activity of all these enzymes in the supernatant is strongly influenced by the compositions of the plant peptones used for cultivation and may vary by a factor 5 or more, depending on the source or raw material of a plant peptone, the manufacturer, the production process, and on the batch. As outlined above, such product variability and dependency on a single supplier are suboptimal for an industrial production process, especially in the pharmaceutical industry.

### Purpose of the invention

The aim of this invention was to provide a liquid medium and/or a liquid feed composition which (i) supports growth of the bacteria and (ii) stimulates secretion of enzymes with proteolytic activity into the culture medium and (iii) reduces product and process variability. It is particularly preferred that the medium stimulates a high volume activity (enzyme activity per culture volume) in the culture supernatant. An additional purpose of the present invention is to provide a new type of culture medium that allows the cultivation of bacteria without addition of animal-derived components and/or plant derived components. The medium should furthermore have as few components as possible, in order to avoid any contamination with undesirable substances and to avoid overly complex influences on the fermentation process. It is especially desired that such culture medium allows for the effective cultivation of the species Hathewaya histolytica (Clostridium histolyticum) and for the production of one or more proteases, e.g. collagenase I, collagenase II, neutral protease and/or clostripain. It is a further object of the present invention to provide a supernatant of a Hathewaya histolytica (Clostridium histolyticum) liquid culture, which may be used for the isolation of the one or more proteases comprised therein. It is also an object of the present invention to provide a process for the production of proteases wherein the degradation of the proteases is low. In addition, the culture medium may provide the following benefits: a) higher batch reliability than culture media comprising animal peptones or plant peptones; b) all components can be obtained in suitable and constant quality from multiple suppliers; c) the activity of collagenase in the supernatant of Hathewaya histolytica (Clostridium histolyticum) is 400 U/L or more (PZ activity).

### DETAILED DESCRIPTION

In a first aspect, the present invention concerns a liquid culture medium comprising, essentially consisting of, or consisting of
2.5 to 100 g/L of yeast extract,
1.0 to 30 g/L of glycine,
1.0 to 35 g/L of arginine,
0.20 to 0.80 g/L of glutamine,
0.20 to 1.0 g/L of serine,
0.3 to 3.0 g/L of threonine,
0.4 to 12 mmol/L magnesium ions (Mg²⁺), e.g. 100 to 3000 mg/L of magnesium sulfate heptahydrate (MgSO₄×7 H₂O),
0.34 to 3.4 mmol/L calcium ions (Ca²⁺), e.g. 50 to 500 mg/L of calciumchloride dihydrate (CaCl₂×2 H₂O),
0.00095 to 0.0152 mmol/L selenium, e.g. 0.25 to 4.00 mg/L sodium selenite pentahydrate (Na₂SeO₃×5 H₂O), and water.

The term “essentially consists of” as used herein shall mean that the composition (a) necessarily includes the listed ingredients and (b) is open to unlisted ingredients that do not materially affect the basic and novel properties of the composition.

The liquid culture medium according to the present invention is suitable as culture medium for bacteria of the genus Hathewaya (Clostridium) and especially as culture medium for Hathewaya histolytica (Clostridium histolyticum; e.g. Clostridium histolyticum G11 and/or ATCC® 21000™). It provides nutrients required for sufficient growth of the bacteria and—optionally in combination with a feed composition according to the invention—for the effective production of one or more proteases, such as e.g. collagenase I, collagenase II, neutral protease and/or clostripain. In an embodiment, by fermentation of Hathewaya histolytica (Clostridium histolyticum) with the inventive culture medium, and optionally with the inventive feed composition, the activity of collagenase in the supernatant is 400 PZ units/L or more, in another embodiment 600 PZ units/L or more, in another embodiment 1200 PZ units/L or more. 1 PZ Unit according to Wuensch catalyzes the hydrolysis of 1 μmol 4-phenylazobenzyl-oxycarbonyl-L-prolyl-L-leucyl-glycyl-L-prolyl-D-arginine (PZ) per minute at 25 °C, pH 7.1 (Wuensch, E. & Heidrich, H. G. (1963) Hoppe-Seyler's Z. Physiol. Chem. 333, 149-51).

The use of lower concentrations than 2.5 g/L of yeast extract is in principle possible, but may reduce bacterial growth and product yield. The use of concentrations higher than 100 g/L of yeast extract is in principle possible, but may not provide much benefit in terms of bacterial growth or secretion of proteases. In some embodiments, the concentration of yeast extract in the liquid culture medium is in the range of 2.5 to 80, 5.0 to 60 or 10 to 30 g/L. In another embodiment, the concentration of yeast extract in the liquid culture medium is in the range of 17 to 25 g/L. In some embodiments, the liquid culture medium comprises 20 g/L yeast extract.

In principle, any yeast extract can be used. Yeast extracts and methods for the preparation are well known in the art. Contrary to animal peptones or plant peptones, yeast extracts are produced by better controlled industrial processes which include fermentation of the yeast in an industrial bioreactor. For this reason, batch reliability is higher than for plant- or animal-derived components. Different batches of yeast extract produced by the same process provide a similar performance in terms of bacterial growth and production of proteases. Furthermore, yeast extracts produced by different processes and/or different producers can substitute each other and provide similar performance in terms of bacterial growth and production of proteases. In summary, yeast extracts provide increased process robustness over animal peptones and plant peptones.

Especially, any commercially available yeast extract may be used. Non-limiting examples are “BD Yeast Extract Technical” (Becton Dickinson and Company, Miami, FL, USA, Art.-No.: 288610), “BD Yeast Extract” (Becton Dickinson, Art.-No.: 212730), “BD Bacto Yeast Extract” (Becton Dickinson, Art.-No.: 212730). “BD Yeast Extract Technical” (Becton Dickinson, Art.-No.: 288610) provides a high yield of collagenases and other proteases and fermentations with this yeast extract are well reproducible. Further suitable yeast extracts are e.g. Difco Yeast Extract, low-dusting (LD) Art.-No.: 210933″, yeast extracts produced by Ohly, the Kerry HY-Yeast series, and the Yeast Extract Art.-No.: A1552 from AppliChem.

If not otherwise mentioned in this text, the yield and concentration of collagenase is measured by the PZ-activity of the supernatant. The activity of neutral protease in the supernatant can be measured e.g. according to Lin, Y.-C. et al. (1969) J. Biol. Chem. 244, 789-93; or Kunitz, M. 1947. Crystalline soybean trypsin inhibitor. II. General properties. J. Gen. Physiol. 30:291-310; or as described in Example 1. The activity of clostripain in the supernatant can be measured e.g. as described in Kzedy, F. J. et al. Biochemistry, 1965, 4, 2303-2308; or as described in Example 2.

The inventive liquid culture medium comprises glycine or its pharmaceutically acceptable salts in a concentration of 1.0 to 30 g/L. The use of lower concentrations than 1.0 g/L of glycine is in principle possible, but may reduce bacterial growth and may result in lower PZ-activity in the supernatant. Higher concentrations of glycine than 30 g/L may have little benefit for cell growth or PZ-activity in the supernatant. In one embodiment, the concentration of glycine in the inventive liquid culture medium is in the range of 5 to 8 g/L. In another embodiment, the concentration of glycine in the liquid culture medium is in the range of 5 to 7 g/L. In some embodiments, the liquid culture medium comprises 6.5 g/L glycine.

The inventive liquid culture medium comprises arginine or its pharmaceutically acceptable salts in a concentration of 1.0 to 35 g/L. The use of lower concentrations than 1.0 g/L of arginine is in principle possible, but may reduce bacterial growth and may result in lower PZ-activity in the supernatant. Higher concentrations of arginine than 35 g/L may have little benefit for cell growth or PZ-activity in the supernatant. In one embodiment, the concentration of arginine in the inventive liquid culture medium is in the range of 5 to 8 g/L. In another embodiment, the concentration of arginine in the inventive liquid culture medium is in the range of 6 to 7 g/L. In some embodiments, the liquid culture medium comprises 6.7 g/L arginine.

The inventive liquid culture medium comprises glutamine or its pharmaceutically acceptable salts in a concentration of 0.20 to 0.80 g/L. Glutamine has a positive influence on bacterial growth and on the PZ-activity in the supernatant. The use of lower concentrations than 0.20 g/L of glutamine is in principle possible, but may reduce bacterial growth and may result in lower PZ-activity in the supernatant. Higher concentrations of glutamine than 0.80 g/L may have little benefit for cell growth or PZ-activity in the supernatant. In one embodiment, the concentration of glutamine in the inventive liquid culture medium is in the range of 0.25 to 0.40 g/L. In another embodiment, the concentration of glutamine in the inventive liquid culture medium is in the range of 0.30 to 0.35 g/L. In some embodiments, the liquid culture medium comprises 0.32 g/L glutamine.

The inventive liquid culture medium comprises serine or its pharmaceutically acceptable salts in a concentration of 0.20 to 1.0 g/L. Serine has a positive influence on bacterial growth, but a negative influence on productivity. Productivity, as defined herein, is the ratio of PZ-activity to bacterial growth. Bacterial growth can be determined by known methods, e.g. by measuring the turbidity. In some embodiments, bacterial growth is determined by measuring the optical density at 600 nm. The use of lower concentrations than 0.20 g/L of serine is in principle possible, but may result in lower bacterial growth. Serine concentrations higher than 1.0 g/L may reduce productivity. In an embodiment, the concentration of serine in the inventive liquid culture medium is in the range of 0.37 to 0.50 g/L. In another embodiment, the concentration of serine in the inventive liquid culture medium is in the range of 0.37 to 0.47 g/L. In some embodiments, the liquid culture medium comprises 0.42 g/L serine.

The inventive liquid culture medium comprises threonine or its pharmaceutically acceptable salts in a concentration of 0.3 to 3.0 g/L. The lower concentrations of threonine within this range provide higher PZ-activity in the supernatant than the higher concentrations within this range. The use of lower concentrations than 0.3 g/L of threonine will result in lower bacterial growth. Higher concentrations of threonine than 3.0 g/L may reduce the productivity. In an embodiment, the concentration of threonine is not higher than 3.0 g/L. In one embodiment, the concentration of threonine in the inventive liquid culture medium is in the range of 0.5 to 1.50 g/L. In another embodiment, the concentration of threonine in the inventive liquid culture medium is in the range of 0.8 to 1.2 g/L. In some embodiments, the liquid culture medium comprises 1 g/L threonine.

The liquid culture medium according to the invention comprises 0.4 to 12 mmol/L magnesium ions (Mg²⁺), e.g. 100 to 3000 mg/L of magnesium sulfate heptahydrate (MgSO₄×7 H₂O). In an embodiment the liquid culture medium according to the invention comprises 1.2 to 6.9 mmol/L magnesium ions (Mg²⁺). In an embodiment, the concentration of magnesium sulfate heptahydrate in the liquid culture medium is in the range of 300 to 1700 mg/L. In another embodiment the liquid culture medium according to the invention comprises 2.8 to 5.3 mmol/L magnesium ions (Mg²⁺). In another embodiment, the magnesium sulfate heptahydrate concentration is in the range of 700 to 1300 mg/L. In some embodiments, the liquid culture medium comprises 995 mg/L magnesium sulfate heptahydrate. The liquid culture medium according to the invention comprises 0.34 to 3.4 mmol/L calcium ions (Ca²⁺), e.g. 50 to 500 mg/L of calcium chloride dihydrate (CaCl₂×2 H₂O). In an embodiment, the liquid culture medium according to the invention comprises 0.68 to 2.0 mmol/L calcium ions (Ca²⁺). In an embodiment, the concentration of calcium chloride dihydrate in the liquid culture medium is in the range of 100 to 300 mg/L. In another embodiment, the liquid culture medium according to the invention comprises 1.0 to 2.0 mmol/L calcium ions (Ca²⁺). In another embodiment, the calcium chloride dihydrate concentration is in the range of 150 to 300 mg/L. In some embodiments, the liquid culture medium comprises 260 mg/L calcium chloride dihydrate. In one embodiment the anions for the magnesium and calcium ions may be selected from the group consisting of chloride, sulfate, phosphate and acetate may be used. In other embodiments magnesium sulfate may be used for magnesium ions and/or calcium chloride may be used for calcium ions. The liquid culture medium according to the invention comprises 0.00095 to 0.0152 mmol/L selenium, e.g. 0.25 to 4.00 mg/L sodium selenite pentahydrate (Na₂SeO₃×5 H₂O). In an embodiment, the liquid culture medium according to the invention comprises 0.0027 to 0.0061 mmol/L selenium. In an embodiment, the concentration of sodium selenite pentahydrate in the liquid culture medium is in the range of 0.70 to 1.60 mg/L. In another embodiment, the liquid culture medium according to the invention comprises 0.0038 to 0.0053 mmol/L selenium. In another embodiment, the sodium selenite pentahydrate concentration is in the range of 1.00 to 1.40 mg/L. In some embodiments, the liquid culture medium comprises 1.17 mg/L sodium selenite pentahydrate. In some embodiments sodium selenite (Na₂SeO₃) or its pentahydrate is used to provide selenium to the inventive liquid culture medium, i.e., the medium comprises sodium selenite. Other selenium comprising compounds or compositions may be used. It is well within the knowledge of the expert in the art to choose suitable forms of selenium. It is well known that the selenium introduced into the media may be in a suitable form that has sufficient bioavailability. This makes the selenium comprised in the media available to the bacteria. Whether a compound or a composition provides sufficient selenium to the media can simply be determined by comparison of the yield of the desired proteases in a fermentation. This yield can be compared with the yield obtained through use of sodium selenite. If necessary, the amounts of compounds or composition other than sodium selenite or its pentahydrate used to provide selenium could be adjusted so that the same or similar results are obtained.

The inventive liquid culture medium as described herein may comprise water, for example, purified water or water for injection. Purified water is water that has been mechanically filtered or processed to remove impurities and make it suitable for use, especially pharmaceutical use. Water for injection is even more purified and is further defined e.g. in the European or United States Pharmacopeia. In one embodiment, the liquid culture medium comprises water in the amount of at least 50, 60, or 70 percent by weight based on the whole weight of the liquid culture medium. In some embodiments, the liquid culture medium comprises water in the amount of at least 78% by weight, or in the amount of at least 90% by weight, or in the amount of at least 95% by weight. In some embodiments, water is the remainder of the liquid culture medium. In some embodiments, the liquid culture medium comprises purified water or water for injection according to the European or United States Pharmacopeia, e.g. in the above specified amounts.

In some embodiments, the liquid culture medium is used for the fermentation of Hathewaya histolytica (Clostridium histolyticum) and the production of at least one protease selected from the group consisting of collagenase I, collagenase II, neutral protease and clostripain.

In some embodiments, the liquid culture medium comprises, essentially consists of, or consists of 10 to 30 g/L of yeast extract, 5 to 8 g/L of glycine, 5 to 8 g/L of arginine, 0.25 to 0.40 g/L of glutamine, 0.37 to 0.50 g/L of serine, 0.50 to 1.50 g/L of threonine, 1.2 to 6.9 mmol/L magnesium ions (Mg²⁺), 0.68 to 2.0 mmol/L calcium ions (Ca²⁺), 0.0027 to 0.0061 mmol/L selenium, and water. In some embodiments, the liquid culture medium comprises, essentially consists of, or consists of 10 to 30 g/L of yeast extract, 5 to 8 g/L of glycine, 5 to 8 g/L of arginine, 0.25 to 0.40 g/L of glutamine, 0.37 to 0.50 g/L of serine, 0.50 to 1.50 g/L of threonine, 300 to 1700 mg/L of magnesium sulfate heptahydrate (MgSO₄×7 H₂O), 100 to 300 mg/L of calcium chloride dihydrate (CaCl₂×2 H₂O), 0.70 to 1.60 mg/L of sodium selenite pentahydrate (Na₂SeO₃×5 H₂O), and water. In some embodiments, the liquid culture medium comprises, essentially consists of, or consists of 17 to 25 g/L of yeast extract, 5 to 7 g/L of glycine, 6 to 7 g/L of arginine, 0.30 to 0.35 g/L of glutamine, 0.37 to 0.47 g/L of serine, 0.8 to 1.2 g/L of threonine, 2.8 to 5.3 mmol/L magnesium ions (Mg²⁺), 1.0 to 2.0 mmol/L calcium ions (Ca²⁺), 0.0038 to 0.0053 mmol/L selenium, and water. In some embodiments, the liquid culture medium comprises, essentially consists of, or consists of 17 to 25 g/L of yeast extract, 5 to 7 g/L of glycine, 6 to 7 g/L of arginine, 0.30 to 0.35 g/L of glutamine, 0.37 to 0.47 g/L of serine, 0.8 to 1.2 g/L of threonine, 700 to 1300 mg/L of magnesium sulfate heptahydrate, 150 to 300 mg/L of calcium chloride dihydrate, 1.00 to 1.40 mg/L of sodium selenite pentahydrate, and water. In some embodiments, the liquid culture medium comprises, essentially consists of, or consists of 20 g/L yeast extract, 6.5 g/L glycine, 6.7 g/L arginine, 0.32 g/L glutamine, 0.42 g/L serine, 1 g/L threonine, 995 mg/L magnesium sulfate heptahydrate, 260 mg/L calcium chloride dihydrate, and 1.17 mg/L sodium selenite pentahydrate, and water (e.g. purified water).

Since the yeast extracts can be prepared in a reproducible way, the inventive liquid culture medium provides a stable and reproducible culture medium with a high batch stability, which provides for a high process robustness for the cultivation of Hathewaya histolytica (Clostridium histolyticum) and the production of proteases, such as e.g. collagenase I, collagenase II, neutral protease and clostripain. In some embodiments, the liquid culture medium is free from any components derived from animals or plants. Such animal or plant derived ingredients may comprise adventitious agents and/or their ingredients may vary between manufacturers and even between different batches of the same manufacturer. Using a liquid culture medium without animal or plant derived ingredients for the cultivation of Hathewaya histolytica (Clostridium histolyticum) and the production of proteases provides more safety for the end user of the proteases and improves reliability of the inventive liquid culture medium in fermentations.

The terms “derived from animals” or “animal derived” or “animal-derived” as used herein shall mean any substance of animal origin. The terms “derived from plants” or “plant derived” or “plant-derived” as used herein shall mean any substance of plant origin. In some embodiments, the terms “derived from animals” or “animal derived” or “animal-derived” as used herein shall mean any substance of animal origin and any substance of non-animal origin processed using one or more animal-derived substances (e.g. animal-derived enzymes). In some embodiments, the terms “derived from plants” or “plant derived” or “plant-derived” as used herein shall mean any substance of plant origin and any substance of non-plant origin processed using one or more plant-derived substances (e.g. plant-derived enzymes). The terms “component” or “ingredient” as used herein shall mean any substance of content of the liquid culture medium or the liquid feed composition.

In some embodiments, the liquid culture medium or the liquid feed composition does not comprise glucose. In some embodiments, the liquid culture medium or the liquid feed composition does not comprise a sugar selected from the group consisting of glucose, galactose, lactose, mannose, raffinose, fucose, sucrose and arabinose. In some embodiments, the liquid culture medium or the liquid feed composition does not comprise any sugar.

In a further embodiment, the liquid culture medium comprises an anti-foaming agent. Anti-foaming agents suppress the development of foam in the culture medium and during fermentation. Any commercially available anti-foaming agent may be used. One suitable anti-foaming agent is XIAMETER™ ACP-1500 (EU) Antifoam Compound from Dow. The anti-foaming agent may be added in an amount suitable to suppress foaming to an acceptable level. Fermentations in small scale may be conducted without the addition of anti-foaming agent. In large-scale fermentations anti-foaming agent may be added.

In some embodiments, the liquid culture medium has a pH value in the range of 6.5 to 8.2. In some embodiments, the pH value of the liquid culture medium is in the range of 7.2 to 8.1 or 7.5 to 8.0. The pH value may be regulated by any known method, e.g. by addition of a buffer with a suitable buffer capacity or by addition of acids or bases. In some embodiments, for example in pre-cultures and in fermentations with supernatants with a volume of about 0.5 liter or less, buffer may be added. For example, a 3-(N-morpholino) propanesulfonic acid buffer (MOPS buffer) may be used. In some embodiments, for example in a fed-batch process and/or in fermentation processes in which the volume of the supernatant is more than about 1 liter, the pH value may be continuously measured and acids or bases (or both in the time course of a cultivation process) are added to keep the pH value in the desired range. For example, phosphoric acid and/or sodium hydroxide may be used. In some embodiments, for example in fermentation processes in which the volume of the supernatant is between about 0.5 and about 1 L, any of the two methods may be used.

In a further embodiment, the liquid culture medium is sterilized. In some embodiments, the composition is free from any self-replicating organism. Sterilization can be achieved by standard methods known to the skilled person, e.g. by heat treatment, such as, for example, autoclaving. In some embodiments, the sterilized liquid culture medium comprises an inoculum of Hathewaya histolytica (Clostridium histolyticum). In some embodiments, the liquid culture medium is free from any self-replicating organism but Hathewaya histolytica (Clostridium histolyticum). In some embodiments, such compositions are prepared by adding an inoculum of Hathewaya histolytica (Clostridium histolyticum) to a sterilized liquid culture medium of the present invention.

In a second aspect, the present invention concerns a liquid feed composition, for example for use as feed in a fed-batch process for the cultivation of Hathewaya histolytica (Clostridium histolyticum), comprising, essentially consisting of, or consisting of
at least 20 g/L of yeast
extract, at least 3 g/L of threonine,
at least 1.5 g/L of serine,
and water.

The term “fed-batch culture” as used herein shall mean an operational technique in biotechnological processes where one or more nutrients are fed (supplied) to a container, e.g. a bioreactor, during cultivation and in which the product(s) remain in the container until the end of the run. For further details on fed-batch processes see e.g. Tsuneo Yamané and Shoichi Shimizu (Fed-batch Techniques in Microbial Processes; Advances in Biochem Eng./Biotechnol 1984, 30:147-194).

The liquid feed composition and the liquid culture medium are different compositions. In some embodiments, the liquid feed composition comprises at least 30, 40, or 50 g/L yeast extract. In some embodiments, the liquid feed composition comprises at least 4, 5, 6, 7, 8, 9, or 10 g/L threonine. In some embodiments, the liquid feed composition comprises at least 2.0, 2.5, 3.0, 4.5, 5.0 g/L serine. In some embodiments, the liquid feed composition comprises, essentially consists of, or consists of 50 to 200 g/L yeast extract, 3 bis 60 g/L threonine and 1.5 to 40 g/L serine, and water. In some embodiments, the liquid feed composition comprises, essentially consists of, or consists of 70 to 130 g/L yeast extract, 3 bis 10 g/L threonine and 1.5 to 10 g/L serine, and water. In some embodiments, the liquid feed composition comprises, essentially consists of, or consists of 100 g/L yeast extract, 5 g/L threonine and 2.5 g/L serine, and water. The inventive liquid feed composition as described herein comprises water, for example purified water or water for injection, in the amount of at least 50, 60, or 65 percent by weight based on the whole weight of the liquid feed composition. In some embodiments, the liquid feed composition comprises water, for example purified water or water for injection, in the amount of at least 70 percent by weight, or at least 80 percent by weight, or at least 95% by weight. In some embodiments, water is the remainder of the liquid feed composition.

In some embodiments, the liquid feed composition has a pH value in the range of 6.5 to 8.2.

In a further embodiment, the liquid feed composition is sterilized. In some embodiments, the composition is free from any self-replicating organism. Sterilization can be achieved by standard methods known to the skilled person, e.g., by heat treatment, such as, for example, autoclaving.

In a third aspect, the present invention concerns a kit of parts comprising a liquid culture medium of the present invention and a liquid feed composition of the present invention. The combination is useful, for example, for the fermentation of Hathewaya histolytica (Clostridium histolyticum; e.g. Clostridium histolyticum G11 and/or ATCC® 21000™) and the production of one or more proteases, e.g. in a fed-batch process.

A fourth aspect of the present invention is a supernatant of a Hathewaya histolytica (Clostridium histolyticum, e.g. Clostridium histolyticum G11 and/or ATCC® 21000™) liquid culture, comprising a liquid culture medium of the present invention and/or a liquid feed composition of the present invention and one or more proteases. The one or more proteases may be selected from the group consisting of collagenase I, collagenase II, neutral protease and clostripain. In some embodiments, the supernatant of a Hathewaya histolytica (Clostridium histolyticum) liquid culture prepared by use of a liquid culture medium and/or a liquid feed composition of the present invention has a collagenase activity of at least 400 PZ Units/L (Wuensch units), or at least 500 PZ Units/L, or at least 600 PZ Units/L, or at least 700 PZ Units/L, or at least 800 PZ Units/L, or at least 900 PZ Units/L, or at least 1000 PZ Units/L, or at least 11000 PZ Units/L, or at least 1200 PZ Units/L.

An embodiment of this aspect of the invention is a culture supernatant of Hathewaya histolytica (Clostridium histolyticum) comprising one or more proteases obtainable by the method of (a) providing a sterilized liquid culture medium according to the invention with an inoculum of Hathewaya histolytica (Clostridium histolyticum), (b) cultivating the bacteria, whereby the bacteria secrete the one or more proteases into the liquid phase; (c) separating solids, e.g. cellular and other particulate matter, from the liquid phase; and thereby obtaining the culture supernatant from Hathewaya histolytica (Clostridium histolyticum) comprising one or more proteases. In some embodiments, the supernatant is obtained from a liquid fed-batch culture. Accordingly, in some embodiments, the supernatant is obtainable by the method of (a) providing a sterilized liquid culture medium according to the invention with an inoculum of Hathewaya histolytica (Clostridium histolyticum), (b) cultivating the bacteria in a fed-batch operation, (c) adding the liquid feed composition of the invention, (d) separating solids, e.g. cellular and other particulate matter, from the liquid phase; and thereby obtaining the culture supernatant from Clostridium histolyticum comprising one or more proteases. In some embodiments, the one or more proteases may be selected from the group comprising collagenase I, collagenase II, neutral protease, and clostripain.

In a fifth aspect, the present invention concerns the use of a liquid culture medium of the present invention for cultivating Hathewaya histolytica (Clostridium histolyticum; e.g. Clostridium histolyticum G11 and/or ATCC® 21000™) and obtaining at least one protease from the culture supernatant, e.g. a protease with collagenase activity, such as collagenase I and/or collagenase II, and/or neutral protease and/or clostripain. In a sixth aspect, the present invention concerns the use of a liquid feed composition of the present invention for cultivating Hathewaya histolytica (Clostridium histolyticum; e.g. Clostridium histolyticum G11 and/or ATCC® 21000™) and obtaining at least one protease from the culture supernatant, e.g. a protease with collagenase activity, such as collagenase I and/or collagenase II, and/or neutral protease and/or clostripain.

In a seventh aspect, the present invention concerns a method comprising the step of cultivating Hathewaya histolytica (Clostridium histolyticum; e.g. Clostridium histolyticum G11 and/or ATCC® 21000™) in a liquid culture medium of the present invention and obtaining at least one protease from the culture supernatant.

In some embodiments, collagenase I, collagenase II, neutral protease and/or clostripain are obtained with the aforementioned method. In some embodiments of the inventive method, the pH value of the culture is controlled such that it is in the range of 6.5 to 8.2. In some embodiments, the pH value of the culture is controlled such that it is in the range of 7.2 to 8.1 or 7.5 to 8.

In some embodiments of the method of the present invention, the cultivation is preferably performed in a fed-batch operation by use of a sterilized liquid feed composition as described herein as feed. In some embodiments of such a fed-batch process, the pH value of the culture is controlled such that it is in the range of 7.2 to 7.8.

In some embodiments, the method according to the present invention comprises the steps of
(a) providing a sterilized liquid culture medium according to the present invention with an inoculum of Hathewaya histolytica (Clostridium histolyticum bacteria; e.g. Clostridium histolyticum G11 and/or ATCC® 21000™);
(b) cultivating the bacteria, optionally adding a liquid feed composition according to the present invention, whereby the bacteria secrete the one or more proteases into the liquid phase;
(c) separating solids, e.g. cellular and other particulate matter, from the liquid phase, thereby obtaining a supernatant;
(d) obtaining the one or more proteases from the supernatant;
thereby producing the one or more proteases from Hathewaya histolytica (Clostridium histolyticum).

The culture or fermentation process may be monitored continuously or at one or more time points during cultivation, e.g. by determining turbidity and/or by determining protease activity in a sample of the culture supernatant (which may be taken at one or more time points during cultivation). In some embodiments, the addition of the liquid feed composition is started when bacterial growth rate has reached the exponential phase. In some embodiments, the addition of the liquid feed composition is started when bacterial growth rate has reached about 40%, 50%, 60%, or 70% of the turbidity maximum. The bacteria may be cultivated for 15 to 19 hours. Since the enzyme ratio changes over time, the cultivation time may be adapted according to the one or more target enzymes. Suitable methods to monitor the cultivation and bacterial growth phases are e.g. disclosed in EP 2 133 415 A1. The one or more proteases may be obtained from the culture supernatant by any known methods, e.g. in crude form or purified. Accordingly, in some embodiments, step (d) may comprise one or more purification steps such as e.g. filtration and/or column chromatography. WO 2020/164721 A1 describes some example procedures for obtaining one or more proteases from a culture supernatant of Hathewaya histolytica (Clostridium histolyticum).

### Examples

### Analytic Methods:

Determination of Wuensch activity in the supernatant has been conducted according to Wuensch, E., Heidrich, H. G.; Hoppe-Seyler's Zeit. Physiol. Chem., 333, 149-51 (1963). 1 PZ unit activity according to Wuensch is the activity that catalyzes the hydrolysis of 1 μmol 4-phenyl-azobenzyloxycarbonyl-L-prolyl-L-leucyl-glycyl-L-prolyl-D-arginine per minute at 25 °C, pH 7.1. All values of Wuensch units herein are given in PZ Units/liter (U/L), i.e. Wuensch units per liter of culture medium, if not otherwise stated.

The activities of clostripain and neutral protease have been determined as described in Examples 1 and 2.

The turbidity was measured off-line in the Mettler Toledo UV-VIS spectrophotometer UV5Nano at 600 nm (OD 600). It is measured against an empty sample (medium without inoculum). If necessary, the solution must be diluted so that the measured value is in the measuring range of up to 0.6 OD 600.

### Materials:

In all experiments Clostridium histolyticum G11 and/or ATCC® 21000™ were used. ATCC® 21000™ is available at the ATCC (American Type Culture Collection). As yeast extract “BD Yeast Extract” (Becton Dickinson, Art.-No.: 288610) was used. In the below described experiments all cultures were inoculated with 2 volume percent of a pre-culture. The pre-culture was prepared by Inoculation of the liquid culture medium with the contents of 1 vial (the vials were defined to contain a minimum of 10⁶ CFU; according to analysis the vials contained 35×10⁶ CFU/Vial.) of lyophilized Hathewaya histolytica (Clostridium histolyticum G11 or ATCC® 21000™) mixed with 1 ml of the liquid culture medium and anaerobic incubation at 37 °C in shake flasks.

As bioreactors Multifors bioreactors (from Infors GmbH, Einsbach, Germany) were used. Further materials, reagents and devices are described in the Examples below.

### Example 1: Activity Assay of Neutral Protease Using a Continuous Photometric Assay with the Substrate N-(3-[Furyl] Acryloyl)-Glycine Leucine Amide (FAGLA)

Principle: The neutral protease from Hathewaya histolytica (or Clostridium histolyticum) catalyzes the hydrolysis of the peptide bond in the substrate FAGLA between the amino acids glycine and leucine. In the photometric assay, substrate conversion was measured continuously by decreasing absorbance at 345 nm. The decrease in absorbance or the resulting negative slope is thus a direct measure of substrate conversion and thus of enzyme activity.

Units definition: One FAGLA unit (1U) is defined as the hydrolysis of 1 μmol of N-[3-(2-furyl) acryloyl]-glycine-L-leucine amide per minute.

Devices: temperature controlled UV-VIS spectrophotometer (e.g. Cary 50, 60 or 100, Agilent), circular shaker (e.g. Scientific Industries, Vortex Genie2), UV cuvettes semi-micro (e.g. Brand, Art. No. 759150), semi-micro quartz cuvettes (e.g. Hellma), PD-10 Desalting Columns (e.g. GE Healthcare, Art. No. 17-0851-01).

Reagents: purified water, Ph. Eur., 2-Morpholinoethanesulfonic acid monohydrate, abbrev. MES (e.g. Merck, Art. No. 7 1.060126), Calcium chloride dihydrate (e.g. Merck, Art. No. 1.02382), Dimethyl sulfoxide for spectroscopy, abbreviation DMSO (e.g. Merck, Art. No. 1.02950), Triton X-100 (e.g. Sigma-Aldrich, Art. No. 23,472-9, 2-propanol (e.g. Merck, Art. No. 1.01040), 1 mol/l Sodium hydroxide solution (Merck, Art. No. 1.09137), FA-Gly-Leu-NH2 (e.g. Bachem, Art. No. 4003615), current reference substance Neutral Protease NB (REF00236), Tris(hydroxymethyl)aminomethane, abbreviation Tris (e.g. Merck, Art. No. 1.08382), 1 mol/l hydrochloric acid (e.g. Merck, Art. No. 1.09057).

### Reagent Solutions:

a) MES buffer pH 6.5:10.67 g (0.05 mol) of 2-morpholinoethanesulfonic acid monohydrate and 0.735 g (0.005 mol) of calcium chloride dihydrate were dissolved in approximately 800 ml of purified water. Then 10 ml of 2-propanol (1% (v/v)) was added to the buffer mixture. The pH was adjusted to 6.5 at room temperature with 1 mol/l sodium hydroxide solution and filled up to 1000 ml with purified water. 0.1 ml of Triton X-100 were added to the buffer while stirring by using a 100 μl piston-stroke pipette to slowly pipette the highly viscous Triton X-100 solution (0.01% (v/v). The detergent was added to the buffer mixture and at the end the tip is dropped into the buffer. The solution was stirred until homogeneity was reached. The obtained buffer solution has a concentration of 0.05 mol/l 2-morpholinoethanesulfonic acid and 0.005 mol/l of calcium chloride.
b) FAGLA substrate solution (concentration=2.5 mmol/l): First, a 0.1M FAGLA solution was prepared (36.9 mg FAGLA dissolved in 1.2 ml dimethyl sulfoxide). Then 1 ml of 0.1 M FAGLA solution was mixed with 39 ml of MES buffer pH 6.5, as described under a).
c) Reference substance: Approximately 12-20 mg of the reference substance Neutral Protease NB (Nordmark Biochemicals, Uetersen, Germany) was weighed and adjusted to a concentration of 10 mg/ml with MES buffer pH 6.5 as described under a). This solution was stored on ice. Further dilutions (within the valid working range, as described below) were prepared from this solution with MES buffer pH 6.5. Approximately 2.5 to 3.6 mg/ml of reference substance was used. The absorbance changes must lie within the valid working range. The analyte solutions were prepared shortly before the determination with MES buffer pH 6.5 at RT and measured promptly. The activity of the reference substance was measured in duplicate for at least one dilution in the valid working range.
d) Tris buffer pH 9.5:0.606 (0.005 mol) tris(hydroxymethyl)aminomethane and 0.37 g (0.0025 mol) calcium chloride dihydrate was dissolved in approximately 400 ml purified water. The pH was adjusted to 9.5 with 1 mol/l hydrochloric acid at room temperature and filled up to 500 ml with purified water. The resulting buffer had a concentration of tris(hydroxymethyl)aminomethane of 0.01 mol/l and of Calcium chloride of 0.005 mol/l).

### Sample Preparation:

At least one duplicate determination was carried out. Depending on the activity, the samples were diluted to be within the valid working range (see below). Samples from the concentrate of a cell-free culture supernatant were re-buffered with a PD-10 column equilibrated with Tris buffer pH 9.5 before measurement.

### Implementation:

The test was performed at +30 °C and a wavelength of 345 nm in a temperature-controlled UV-VIS spectrophotometer. The absorbance change at 345 nm was determined for a time interval of 5 min.

The FAGLA substrate solution was tempered for approx. 30 min in a water bath at +30 °C before the actual measurement. Alternatively, the semi-micro cuvettes incl. 0.8 ml substrate solution can be tempered directly in the UV-VIS photometer for at least 10 min before measurement. Before starting a measuring series, the photometer was calibrated against MES buffer pH 6.5 without substrate solution. For the measurement, 0.8 ml of tempered substrate solution was placed in the cuvettes. Then 0.2 ml sample solution was added, immediately premixed by drawing up with the pipette (approx. three piston strokes) and the entire reaction mixture was mixed with a disposable stirring spatula. Then the measurement was started immediately. The final volume in the test was 1 ml. At least one duplicate determination was carried out on each sample. The specified substrate concentration of 2 mmol/l was below the saturation value, i.e. the measured specific activity depends on the substrate concentration. Therefore, the substrate concentration in the assay must be strictly adhered to and only activities determined at identical substrate concentrations are comparable.

### Valid Working Range:

A prerequisite for an evaluable activity measurement is an absorbance change per minute (Δ A 345 nm/min) in the valid working range from −0.01 to −0.035 and the course of the negative slope must be linear. If the values are too high (Δ A 345 nm/min), stronger dilutions must be used and if the values are too low, lower dilutions are to be used.

### Evaluation:

The current reference substance was evaluated. First, a target value and acceptance criteria were defined. The activities of the reference substance and the samples from the mean values of the duplicate determination were calculated. To calculate the negative absorbance change (AA/min) of the measurement, values from 0 to 5 min were evaluated. Acceptance criteria for evaluation: Δ A 345 nm/min of all measured values must lie in the linear working range. The maximum deviation may be ≤8% of the mean value. If there were major deviations, the measurements were repeated.

The following formula was used to calculate the activity of samples with unknown concentration:

$$Neutral Protease Activity [ \text{U/} ml ] = \frac{Δ A * V * VF}{ε * v}$$

The following formula was used to calculate the activity of samples with known weights:

$$Neutral Protease Activity [ \text{U/} mg ] = \frac{Δ A * V * VF}{ε * v * m}$$

Δ A=Absorption change per minute at 345 nm VF
=Dilution factor of the sample
V=1 ml (total volume)
v=0.2 ml (sample volume)
ε=−0.317 mM⁻¹ cm⁻¹ absorption coefficient of FAGLA m
=sample weight in mg

### Example 2: Determination of Clostripain Activity in Culture Supernatant from Clostridium histolyticum

### Principle:

The enzyme clostripain catalyzes the hydrolysis of benzoyl-L-arginine ethyl ester (BAEE) in the presence of calcium and the reducing agent dithiothreitol (DTT), as measured photometrically at 255 nm (Kezdy F J, Lorand L, Miller K D. Titration of active centers in thrombin solutions. Standardization of the enzyme. Biochemistry 1965; 4:2302-8). The increase in absorbance per unit time is a direct measure of enzyme concentration when the reaction is of pseudo-zero order, that is, when it is constant. One unit is the amount of enzymatic activity that catalyzes the hydrolysis of 1 μmol BAEE per minute at 25 °C and pH 7.8 in the presence of 2.5 mM DTT.

Devices: Spectrophotometer Cary 50 (Varian) with temperature-controlled cuvette block Vortexer, Rotator, Quartz cuvettes, d=10 mm.

Reagents: Purified water, Ph. Eur. and USP, Dithiothreitol (DTT) (Serva, Art. No. 20710), Na-Benzoyl-L-arginine-ethyl ester HCl (BAEE) (Serva, Art. No. 14600), Sodium dihydrogen phosphate monohydrate (Merck, Art. No. 6346), 1 N Sodium hydroxide solution (Merck, Art. No. 09137), Calcium acetate hydrate (Kraft, Art. No. 15208)

Reagent solutions: 7.5 mM DTT solution: 57.8 mg DTT were dissolved to 50 ml purified water. The solution was prepared fresh daily. 1.5 mM BAEE solution: 51.8 mg BAEE were dissolved to 100 ml purified water. The solution was prepared fresh daily. 75 mM sodium phosphate buffer, pH 7.8:10.35 g of sodium dihydrogen phosphate was dissolved in approximately 800 ml of purified water and the pH was adjusted accurately to pH 7.8 with sodium hydroxide solution at room temperature. The solution was then filled up to 1000 ml with purified water.

Enzyme solvents: Solution A (without activation): 1.0 mM Ca(OAc)₂: 40 mg calcium acetate hydrate were dissolved to 250 ml purified water. Solution B (with activation): 1.0 mM Ca(OAc)₂ with 5 mM DTT: 38.4 mg DTT was dissolved with solution A to 50 ml. Solution C (with activation): 1.0 mM Ca(OAc)₂ with 2.5 mM DTT: 19.2 mg DTT was dissolved with solution A to 50 ml. The solutions were prepared fresh daily.

Reference substance: For each analysis, a Collagenase NB1 (Nordmark Biochemicals, Uetersen, Germany) as reference substance for identity was also analyzed.

Sample Preparation: The protein concentration in the cell-free culture supernatant was determined by measuring absorbance at 280 nm by spectrophotometry. Three samples of the cell-free culture supernatant where taken and each sample was diluted with Solution B to a concentration of 20 mg of the sample per ml of Solution B.

Each of those diluted samples was split as follows:

$$\begin{matrix}per sample \\ weight\end{matrix} \begin{matrix}↗ & 500 μl sample + 500 μl Solution B ( Sample X ) 500 \\ ↘ & μl sample + 500 μl Solution A ( Sample Y )\end{matrix}$$

Sample X and Sample Y are incubated between 40 minutes and 4 hours at +4 °C and then

100 μl of Sample X are further diluted with 900 μl of Solution C and 200 μl of Sample Y are further diluted with 200 μl of Solution A to obtain Samples X and Y as ready for measurement (“Sample Xm” and “Sample Ym”).

### Implementation:

The activated Sample Xm and the non-activated Sample Ym were measured in parallel. In addition a zero measurement was performed. The following amounts of reagents (pre-tempered to 25 °C) were pipetted into quartz cuvettes:

| | | for | for |
|---|---|---|---|
| | Zero | Sample Ym | Sample Xm |
| Reagent | measurement | (not activated) | (activated) |
| NaH₂PO₄ buffer, pH 7.8 | 1.0 ml | 1.0 ml | 1.0 ml |
| 7.5 mM DTT | — | — | 1.0 ml |
| 1.5 mM BAEE | 1.0 ml | 1.0 ml | 1.0 ml |
| Purified water | 1.1 ml | 1.0 ml | — |
| Sample Xm | — | — | 0.1 ml |
| Sample Ym | — | 0.1 ml | — |

Sample Xm and Sample Ym, respectively, were added last, the content of the cuvettes was mixed and the change in absorbance was measured immediately after mixing at 255 nm for 5 min at 25 °C. The slope AA 255 nm/min was evaluated in the linear region of the curve. The measured slopes of the absorbance at 255 nm per minute of the samples must be linear over the entire 5 minutes, otherwise dilutions must be adapted.

### Calculation of the Activity:

The following formula was used to calculate the activity:

$$Clostripain activity [ \text{U/} ml ] = \frac{Δ {A}_{255 n m} * {V}_{G} * dilution factor}{0.81 * {V}_{P}}$$

ΔA_{255 nm}—slope per minute at 255 nm
V_{G}—total volume used in the test (V_{G}=3.1 ml)
V_{P}—volume of sample used in the test (V_{P}=0.1 ml)
0.81—molar absorption difference of BAEE against benzoyl-L-arginine at 255 nm, i.e. 810 M⁻¹ cm⁻¹
dilution factor: factor of all dilutions beginning with the samples of the cell-free culture supernatant until the measurement

### Example 3: Preparation of a Liquid Culture Medium

20 g/L of yeast extract, 6.5 g/L of glycine, 6.7 g/L of arginine, 0.32 g/L of glutamine, 0.42 g/L of serine, 1.00 g/L of threonine, 995 mg/L of magnesium sulfate heptahydrate (MgSO₄×7 H₂O), 260 mg/L of calcium chloride dihydrate (CaCl₂×2 H₂O) and 1.17 mg/L of sodium selenite pentahydrate (Na₂SeO₃×5 H₂O) and 10 mg of a silicone-based anti-foaming agent (XIAMETER™ ACP-1500 (EU) Antifoam Compound) were added to a 1 liter flask pre-filled with about 600 ml purified water and the flask was subsequently filled with purified water under stirring up to an overall volume of the liquid culture medium of one liter. Finally the liquid culture medium was sterilized in an autoclave. After sterilization, the pH was adjusted to 7.5 by addition of a 10 M sodium hydroxide solution in water.

### Example 4: Preparation of a Liquid Culture Medium for Pre-Cultures

20 g of yeast extract, 6.5 g of glycine, 6.7 g of arginine, 0.32 g of glutamine, 0.42 g of serine, 1.00 g of threonine, 995 mg of magnesium sulfate heptahydrate (MgSO₄×7 H₂O), 260 mg of calcium chloride dihydrate (CaCl₂×2 H₂O), 1.17 mg of sodium selenite pentahydrate (Na₂SeO₃×5 H₂O) and 41.86 g 3-(N-Morpholino) propanesulfonic acid buffer and 10 mg of a silicone-based anti-foaming agent (XIAMETER™ ACP-1500 (EU) Antifoam Compound) were added to a 1 liter flask pre-filled with about 600 ml purified water. The flask was subsequently filled with purified water under stirring up to an overall volume of the liquid culture medium of one liter and the pH was adjusted to 7.9 by addition of a 10 M sodium hydroxide solution in water. Finally the liquid culture medium was sterilized in an autoclave.

### Example 5: Preparation of a Liquid Feed Composition

100 g of yeast extract, 5 g of threonine and 2.5 g of serine were added to a 1 liter flask pre-filled with about 600 ml purified water and the flask was subsequently filled with purified water under stirring up to an overall volume of the liquid culture medium of one liter. Finally the liquid culture medium was sterilized in an autoclave.

### Example 6: First Pre-Culture

A 100 mL Erlenmeyer flask was filled with 100 mL medium according to Example 4. About 1 mL of the medium was removed from the flask and the contents of one vial of lyophilized Clostridium histolyticum G11 (about 35×10⁶ CFU/Vial) or ATCC® 21000™ was mixed therewith. The medium was inoculated with this mixture. The culture thus prepared was grown without shaking for 24 hours (±1 hour). Growth took place either in an anaerobic pot with exclusion of oxygen (Anaerocult, manufacturer Merck) in an incubator or in an anaerobic workbench with anaerobic gas atmosphere (nitrogen 85%, carbon dioxide 10% and hydrogen 5%). The ambient temperature was kept at 37 °C.

### Example 7: Second Pre-Culture

For the fermentation in the 1 L bioreactor, a 100 mL Erlenmeyer flask was filled with 100 ml medium according to Example 4. The medium was inoculated with 5 ml of the first pre-culture (which corresponds to 5% of the second pre-culture medium volume). The culture was grown without shaking for 12 hours (±0.5 hours). Growth took place under anaerobic conditions as disclosed in Example 6 and at an ambient temperature of 37 °C. For the fermentation in the 30 L bioreactor, the second pre-culture was prepared in a 1000 mL flask filled with 1000 ml medium according to Example 4. The medium was inoculated with 50 ml of the first pre-culture (which corresponds to 5% of the second pre-culture medium volume). The culture was grown without shaking for 12 hours (±0.5 hours). Growth took place under anaerobic conditions as disclosed in Example 6 and at an ambient temperature of 37 °C.

### Example 8: Main Culture in a Bioreactor

For the fermentation in the 1 L bioreactor, the start volume of the medium (0.6 L) was inoculated with 12 mL of the second pre-culture (which corresponds to 2% of the medium volume). For the fermentation in the 30 L bioreactor, the start volume of the medium (27 L) was inoculated with 540 mL of the second pre-culture (which corresponds to 2% of the medium volume).

The fermentation parameters for the respective system and the respective scale are shown in the table below:

TABLE 1
Parameter for fermentation in two different bioreactors

Bio reactor

| Parameter | unit | Multifors (1 L) | Techfors (30 L) |
|---|---|---|---|
| Volume of the | L | 0.6 | 27 |
| liquid culture | | | |
| medium (at the | | | |
| beginning) | | | |
| Volume of | mL | 12 | 540 |
| inoculation | | | |
| with second | | | |
| pre-culture | | | |
| Feed rate for | g/h | 25 | 916 |
| the liquid | | | |
| feed | | | |
| composition | | | |
| Nitrogen | L/min | 0.05 | 1.83 |
| atmosphere | vvm | 0.083 | 0.083 |
| Stirrer | rpm | 330 | 165 |
| pH value | — | 7.5 | 7.5 |
| Acids and | — | 10% H₃PO₄, | 10% H₃PO₄, |
| bases | | 5 mM NaOH | 5 mM NaOH |
| used for pH- | | | |
| control | | | |
| Temperature | °C | 37 | 37 |

The liquid culture medium was introduced into the bioreactor and sterilized (the Multifors in an autoclave; the Techfors in the reactor itself). Subsequently the bioreactor was flushed with nitrogen and stirred for at least 3 h before inoculation. 2 volume percent of inoculum of were used. The corresponding amount of the second pre-culture of Example 7 was removed from the second pre-culture using a sterile syringe and the main culture was inoculated via the inoculation port of the bioreactor. On a 30 L scale, the pre-culture was pumped into the bioreactor via a hose (the container with the pre-culture was placed on a scale in order to determine the correct amount of inoculum). The culture time was between 15 and 19 hours, mainly 17 hours and addition of the liquid feed composition according to Example 5 was started after 9 hours of fermentation.

The following table shows the amounts of proteases determined in the supernatant with Clostridium histolyticum G11:

| | 1 L | 30 L |
|---|---|---|
| Collagenase activity [U/mL] | 1.140 | 1.095 |
| Clostripain activity [U/mL] | 4.4 | 5.4 |
| Neutral protease activity [U/mL] | 0.037 | 0.044 |

It can clearly be seen that the scale up for this process was successful and can be achieved without significant loss of activity for any of collagenase, clostripain and neutral protease.

The following table shows the amounts of proteases determined in the supernatant with Clostridium histolyticum ATCC® 21000™:

| | | | Neutral |
|---|---|---|---|
| | Collagenase | Clostripain | Protease |
| Culture supernatant | U/ml | U/ml | U/ml |
| First pre-culture | 0.463 | — | — |
| Second pre-culture | 0.403 | — | — |
| Main culture in 30 L | 0.310 | — | — |
| bioreactor 13 h | | | |
| Main culture in 30 L | 0.354 | 22.8 | 0.119 |
| bioreactor 15 h | | | |
| Main culture in 30 L | 0.425 | 26.8 | 0.125 |
| bioreactor 17 h | | | |
| Main culture in 30 L | 0.443 | 30.7 | 0.146 |
| bioreactor 19 h | | | |

It can clearly be seen that the large-scale production of enzymes was successful and can be achieved with different strains of Clostridium histolyticum.

1. A liquid culture medium comprising
2.5 to 100 g/L of yeast extract,
1.0 to 30 g/L of glycine,
1.0 to 35 g/L of arginine,
0.20 to 0.80 g/L of glutamine,
0.20 to 1.0 of serine,
0.3 to 3.0 g/L of threonine,
0.4 to 12 mmol/L magnesium ions (Mg²⁺),
0.34 to 3.4 mmol/L calcium ions (Ca²⁺), 0.00095 to 0.0152 mmol/L selenium,
and water.

2. A liquid culture medium according to claim 1 comprising
2.5 to 100 g/L of yeast extract,
1.0 to 30 g/L of glycine,
1.0 to 35 g/L of arginine,
0.20 to 0.80 g/L of glutamine,
0.20 to 1.0 of serine,
0.3 to 3.0 g/L of threonine,
100 to 3000 mg/L of magnesium sulfate heptahydrate, 50 to 500 mg/L of calcium chloride dihydrate,
0.25 to 4.00 mg/L sodium selenite pentahydrate, and water.

3. The liquid culture medium according to claim 2 comprising 10 to 30 g/L of yeast extract,
5 to 8 g/L of glycine,
5 to 8 g/L of arginine,
0.25 to 0.40 g/L of glutamine,
0.37 to 0.50 g/L of serine,
0.50 to 1.50 g/L of threonine,
300 to 1700 mg/L of magnesium sulfate heptahydrate, 100 to 300 mg/L of calcium chloride dihydrate,
0.70 to 1.60 mg/L of sodium selenite pentahydrate, and water.

4. The liquid culture medium according to claim 3 comprising 17 to 25 g/L of yeast extract,
5 to 7 g/L of glycine,
6 to 7 g/L of arginine,
0.30 to 0.35 g/L of glutamine,
0.37 to 0.47 g/L of serine,
0.8 to 1.2 g/L of threonine,
700 to 1300 mg/L of magnesium sulfate heptahydrate, 150 to 300 mg/L of calcium chloride dihydrate,
1.00 to 1.40 mg/L of sodium selenitepentahydrate, and water.

5. The liquid culture medium according to claim 1, wherein it is free from any components derived from animals or plants.

6. The liquid culture medium according to claim 1, wherein it has a pH value in the range of 6.5 to 8.2.

7. A sterilized liquid culture medium according to claim 1.

8. The liquid culture medium according claim 1, wherein it additionally comprises an inoculum of Hathewaya histolytica (Clostridium histolyticum).

9. A liquid feed composition for use as feed in a fed-batch process for the cultivation of Hathewaya histolytica (Clostridium histolyticum) comprising
at least 20 g/L of yeast extract, at least 3 g/L of threonine,
at least 1.5 g/L of serine and water.

10. The liquid feed composition according to claim 9, wherein it has a pH value in the range of 6.5 to 8.2.

11. A sterilized liquid feed composition according to claim 9.

12. Kit of parts comprising a liquid culture medium comprising:
2.5 to 100 g/L of yeast extract,
1.0 to 30 g/L of glycine,
1.0 to 35 g/L of arginine,
0.20 to 0.80 g/L of glutamine,
0.20 to 1.0 of serine,
0.3 to 3.0 g/L of threonine,
0.4 to 12 mmol/L magnesium ions (Mg²⁺),
0.34 to 3.4 mmol/L calcium ions (Ca²⁺),
0.00095 to 0.0152 mmol/L selenium, and water; and
a liquid feed composition according to claim 9.

13. Use of a liquid culture medium of claim 1 or a liquid feed composition of claim 9 for cultivating Hathewaya histolytica (Clostridium histolyticum) and obtaining at least one protease from the culture supernatant.

14. Method comprising the steps of:
cultivating Hathewaya histolytica (Clostridium histolyticum) in a liquid culture medium comprising:
2.5 to 100 g/L of yeast extract,
1.0 to 30 g/L of glycine,
1.0 to 35 g/L of arginine,
0.20 to 0.80 g/L of glutamine,
0.20 to 1.0 of serine,
0.3 to 3.0 g/L of threonine,
0.4 to 12 mmol/L magnesium ions (Mg²⁺),
0.34 to 3.4 mmol/L calcium ions (Ca²⁺),
0.00095 to 0.0152 mmol/L selenium, and water; and
obtaining at least one protease from the culture supernatant.

15. Method according to claim 14, wherein the cultivation is performed in a fed-batch operation by use of a liquid feed composition comprising:
at least 20 g/L of yeast extract, at least 3 g/L of threonine;
at least 1.5 g/L of serine and water.
